(19) United States
(12) Patent Application Publication
(10) Pub. No.: US 2010/0074816 A1
(43) Pub. Date: Mar. 25, 2010

(54) Beverage dispenser

(75) Inventors: Tomoharu Nakano, Tokyo (JP); Tetsuo Takeuchi, Chiba (JP); Hiroshi Furuichi, Tokyo (JP); Hideki Tachibana, Kanagawa (JP); Kazuo Furuichi, Kanagawa (JP)

Correspondence Address: DRINKER BIDDLE & REATH (DC), 1500 K STREET, N.W., SUITE 1100, WASHINGTON, DC, 20005-1209, US

(73) Assignee: SUNTORY HOLDINGS LIMITED, Osaka (JP)

(21) Appl. No.: 12/591,400

(22) Filed: Nov. 18, 2009

Related U.S. Application Data
(62) Division of application No. 10/586,474, filed on Jul. 18, 2006.

(30) Foreign Application Priority Data
Jan. 19, 2004 (JP) 2004-011250

(51) Int. Cl. A61L 2/04

(52) U.S. Cl. 422/307

(57) ABSTRACT

A beverage dispenser (10) supplies a beverage from a beverage container (12) through a piping system, wherein a device is provided for sterilizing, by heat, said piping system and a connection portion (61) of said beverage container connected to said piping system. A beverage dispenser further supplies a beverage from a beverage container (12) through a piping system and a cock (32), wherein a device is provided for sterilizing by heat said piping system and said cock (32). Thus, the piping system in the dispenser is heat-sterilized.

### TECHNICAL FIELD

The present invention relates to a dispenser for supplying a beverage including drinking water.

### BACKGROUND ART

There have been placed in the market a variety of types of dispensers for supplying a beverage, for example drinking water, i.e., water servers. Accompanying an increase in the user's concern regarding drinking water, however, it is required to maintain the safety of the drinking water and to enhance the quality, from the taste viewpoint, of the drinking water. Referring to ensuring safety of the drinking water in the case of, for example, a dispenser for feeding tap water, the tap water itself has been sterilized to some extent with chlorine added into the tap water for the purpose of sterilization. Therefore, microorganisms in the tap water are prevented from propagating, and there is no problem. In the case of drinking water such as mineral water and the like, however, no chlorine and the like has been added to the drinking water for sterilization, and propagation of microorganisms in the drinking water becomes a serious problem.

Propagation of microorganisms in drinking water is harmful to the human body if the microorganisms are pathogenic. Even if they are not pathogenic, the microorganisms cause the drinking water to have an offensive taste and an offensive odor and often becoming a cause of turbidity of the drinking water. Microorganisms hardly propagate in the dispenser if the drinking water is supplied consecutively. However, microorganisms may propagate if the drinking water stays in the dispenser for extended periods of time as when the dispenser is left unused in an office during the night or during the weekends. Besides, when used for extended periods of time in the dispenser, flora of microorganisms often propagate gradually.

In order to suppress the propagation of microorganisms in the dispenser, attempts have heretofore been made to pour a germicide or hot water of a high temperature from the outer side of the dispenser into a piping system in the dispenser to circulate it therethrough, or to provide a germ-filtering device in the dispenser. From the standpoint of cost and maintenance, further, attempts have been made as taught in, for example, Japanese Patent No. 3387526 to heat-sterilize a specific portion of the piping system by arranging a heater in the specific portion of the piping system of the dispenser that offers the drinking water from a container filled with the drinking water, without providing any particular sterilizer/filter device.

FIG. 6 is a diagram schematically illustrating another dispenser 100 according to a prior art such as Japanese Unexamined Patent Publication (Kokai) No. 11-190577. Referring to FIG. 6, a container 120 arranged in a refrigerator 110 and filled with the drinking water is connected to a hot water tank 130 heated by a heater 150 and to a cold water tank 140 cooled by a cooler 160 through a pipe 210 and branched pipes 220, 280. As shown, hot water in the hot water tank 130 can be used by operating a hot water cock 310 provided in a pipe 230 and, similarly, cold water in the cold water tank 140 can be used by operating a cold water cock 320 provided in a pipe 250. A hot water introduction pipe 290 extending from the hot water tank 130 works to introduce therein the drinking water of which the volume has increased due to the heating in the hot water tank 130 so that the hot water is prevented from reversely flowing into the branched pipe 220 etc. Further, the hot water or the cold water is more reliably prevented from reversely flowing by a check valve 215 provided in the pipe 210. A communication pipe 240 extending from the hot water tank 130 is communicated with the cold water tank 140 through a circulation valve 350 and a circulation pump 400. For example, at night, the circulation valve 350 is opened and the circulation pump 400 is driven whereby the hot water in the hot water tank 130 is circulated through the communication pipe 240, the cold water tank 140, pipe 220 and pipe 280 so as to heat-sterilize them. In this case, too, the piping system inclusive of the tanks can similarly be heated and sterilized without providing any particular sterilizing/filtering device (see, for example, Japanese Patent No. 3387526 and Japanese Unexamined Patent Publication (Kokai) No. 11-190577).

Here, however, when in use, the container filled with the drinking water taught in the above patent documents is arranged in a dedicated refrigerator. The container is arranged in such a manner that a connection portion, that has been provided for the container, is pierced and inserted by an end of the pipe extending into the refrigerator. The drinking water in the container is cooled by the refrigerator and is maintained at a relatively low temperature of, for example, not higher than 10° C. Therefore, even if various germs that have been adhered onto the connection portion infiltrate at the time of inserting the pipe in the connection portion, it is unlikely that various germs propagate in the container since the temperature in the container is relatively low.

However, the container filled with the drinking water as a whole is arranged in the refrigerator and is maintained cool in the refrigerator until the drinking water in the container is almost all used, causing a problem of consuming large amounts of electric power for cooling the container. Further, if the dispenser is provided with a refrigerator, the cost of production rises, the dispenser becomes bulky and the transportation is difficult.

To solve the above problem, the present inventors have conducted a keen study and have learned that a dispenser capable of supplying hygienic drinking water can be obtained, even without using a refrigerator, by heat-sterilizing the piping systems of the dispenser, by excluding, from the dispenser, the hot water introduction pipes which are portions through which various germs may infiltrate, and by heat-sterilizing the connection portion of the water container and the cocks through which various germs may infiltrate, and have accomplished the present invention.

### DISCLOSURE OF THE INVENTION

It is therefore an object of the present invention to provide a beverage dispenser capable of maintaining sterilizing performance for a beverage despite of omitting the refrigerator.

In order to achieve the above object according to a first aspect of the invention, there is provided a beverage dispenser for supplying a beverage including drinking water from a beverage container through a piping system, wherein a device for sterilizing by heat the piping system and a connection portion of the beverage container connected to the piping system is provided.

According to the first aspect, a cooling device for cooling the beverage container is omitted, e.g., a refrigerator is omitted, making it possible to lower the running cost of the dispenser and to lower the cost of production. Further, the dispenser is realized in a small size facilitating the transportation thereof. The piping system and the connection portion are sterilized by a device that effects sterilization by heat. When the dispenser is in use, a hygienic beverage can be served. Further, when the beverage container is installed in the dispenser while piercing and inserting part of the piping system in the connection portion, it is probable that various germs that have been adhered on the connection portion may adhere on the piping system and may infiltrate into the beverage container at the time of installation. In the first aspect, however, the connection portion is sterilized by heat, and various germs adhered on the connection portion are prevented from infiltrating into the beverage container and from propagating therein.

According to a second aspect of the invention, there is provided a beverage dispenser for supplying a beverage including drinking water from a beverage container through a piping system, wherein:

the piping system is provided with a storage tank for storing the beverage;

the storage tank includes at least a cold beverage tank having a cooling device and a hot beverage tank having a heating device; and

a device for sterilizing by heat the piping system, the cold beverage tank, the hot beverage tank and a connection portion of the beverage container connected to the piping system is provided.

In the second aspect, a cooling device for cooling the beverage container is omitted, e.g., a refrigerator is omitted, making it possible to lower the running cost of the dispenser and to lower the cost of production. Further, the dispenser is realized in a small size facilitating the transportation thereof. The piping system, the cold beverage tank and the hot beverage tank are sterilized by a device that effects the sterilization by heat. When the dispenser is in use, a hygienic hot beverage or cold beverage can be served. Further, when the beverage container is installed in the dispenser while piercing and inserting part of the piping system in the connection portion, it is probable that various germs that have been adhered on the connection portion may adhere on the piping system and may infiltrate into the beverage container at the time of installation. In the second aspect, however, the connection portion is sterilized by heat, and various germs adhered on the connection portion are prevented from infiltrating into the beverage container and from propagating therein.

A third aspect is as set forth in the second aspect, wherein the device for sterilizing by heat causes the hot beverage to arrive at the connection portion of the beverage container connected to the piping system.

When the beverage container is installed in the dispenser while piercing and inserting part of the piping system in the connection portion, it is probable that various germs that have been adhered on the connection portion may adhere on the piping system and may infiltrate into the beverage container at the time of installation. In the third aspect, however, the connection portion is sterilized by the hot beverage, and various germs are prevented from infiltrating into the beverage container and from propagating therein. In the third aspect, the distance between the circulation passage through which the hot beverage circulates and the connection portion, is such that the hot beverage that is circulating arrives at the connection portion but does not arrive at the beverage container.

A fourth aspect is as set forth in the third aspect, wherein the device for sterilizing by heat causes the hot beverage to arrive at the connection portion of the beverage container connected to the piping system by utilizing the volumetric expansion of the hot beverage.

That is, the fourth aspect makes it relatively easy to sterilize the connection portion.

According to a fifth aspect of the invention, there is provided a beverage dispenser for supplying a beverage including drinking water from a beverage container through a piping system, wherein:

the piping system is provided with a storage tank for storing the beverage;

the storage tank includes at least a cold beverage tank having a cooling device and a hot beverage tank having a heating device;

the hot beverage in the hot beverage tank is caused to arrive at a connection portion of the beverage container connected to the piping system, the cold beverage tank and the hot beverage tank are connected together through a communication pipe; and

a device for sterilizing by heat the piping system, the cold beverage tank, the hot beverage tank and the connection portion of the beverage container connected to the piping system by circulating the hot beverage in the hot beverage tank through the piping system, the cold beverage tank and the hot beverage tank using the communication pipe is provided.

In the fifth aspect, a cooling device for cooling the beverage container is omitted, e.g., a refrigerator is omitted, making it possible to lower the running cost of the dispenser and to lower the cost of production. Further, the dispenser is realized in a small size facilitating the transportation thereof. The piping system, the cold beverage tank, the hot beverage tank and the connection portion are sterilized by a device that effects the sterilization by heat. When the dispenser is in use, a hygienic hot beverage or cold beverage can be served. Further, when the beverage container is installed in the dispenser while piercing and inserting part of the piping system in the connection portion, it is probable that various germs that have been adhered on the connection portion may adhere on the piping system and may infiltrate into the beverage container at the time of installation. In the fifth aspect, however, the connection portion is sterilized by heat, and various germs adhered on the connection portion are prevented from infiltrating into the beverage container and from propagating therein. In the fifth aspect, the distance between the circulation passage through which the hot beverage circulates and the connection portion, is such that the hot beverage arrives at the connection portion but does not arrive at the beverage container.

A sixth aspect is as set forth in any one of the first to fifth aspects, wherein absorbing means is provided between the beverage container and the hot beverage tank to absorb a rise in the water level caused by the volumetric expansion of the hot beverage in the hot beverage tank.

In the sixth aspect, even when the hot water introduction pipe and/or the check valve of the prior art are not provided for the dispenser, the rise in the water level caused by the volumetric change is absorbed by the absorbing means when the volume of the hot beverage in the hot beverage tank increases by being heated by the heating device. Therefore, the hot beverage is prevented from reversely flowing into the beverage container to an excess degree, and the pressure in the hot beverage tank is maintained within a predetermined range. The absorbing means may be a pipe in the form of, for example, a continuous coil, or may simply be a pipe that is extended between the beverage container and the hot beverage tank. Or, the absorbing means may be another cooling device provided between the beverage container and the hot beverage tank.

A seventh aspect is as set forth in any one of the second to sixth aspects, wherein the cold beverage tank and the hot beverage tank are connected together through a communication pipe via a circulation pump and an electromagnetic valve, and a timer is provided to operate the electromagnetic valve and the circulation pump at regular intervals.

In the seventh aspect which uses the timer, the cold beverage in the cold beverage tank and the hot beverage in the hot beverage tank are used in a time zone in which the dispenser is used relatively highly frequently, such as during the day time, and the hot beverage is circulated in a time zone in which the dispenser is used relatively lowly frequently, such as at night, to sterilize the piping system.

According to an eighth aspect of the invention, there is provided a beverage dispenser for supplying a beverage including drinking water from a beverage container through a piping system, wherein:

the piping system is provided with a storage tank, for storing the beverage, and with a cock;

the storage tank includes at least a cold beverage tank having a cooling device and a hot beverage tank having a heating device; and

a device for sterilizing by heat the piping system, the cock, the cold beverage tank, the hot beverage tank and a connection portion of the beverage container connected to the piping system is provided.

In the eighth aspect, a cooling device for cooling the beverage container is omitted, e.g., a refrigerator is omitted, making it possible to lower the running cost of the dispenser and to lower the cost of production. Further, the dispenser is realized in a small size facilitating the transportation thereof. The piping system, the cock, the cold beverage tank, the hot beverage tank and the connection portion are sterilized by the device that effects the sterilization by heat. When the dispenser is in use, a hygienic hot beverage or cold beverage can be served. In the eighth aspect, further, not only the piping system but also the cock for supplying the beverage are sterilized by heat, and various germs are prevented from infiltrating into the dispenser through the beverage-pouring port.

A ninth aspect is as set forth in the eighth aspect, wherein the device for sterilizing by heat causes the hot beverage in the hot beverage tank to arrive at a connection portion of the beverage container connected to the piping system, the cold beverage tank and the hot beverage tank are connected together through a communication pipe, and the hot beverage in the hot beverage tank is circulated through the piping system, the cock, the cold beverage tank and the hot beverage tank by using the communication pipe.

When the beverage container is installed in the dispenser while stab-inserting part of the piping system in the connection portion, it is probable that various germs that have been adhered on the connection portion may adhere on the piping system and may infiltrate into the beverage container at the time of installation. In the ninth aspect, however, the connection portion is sterilized by the hot beverage, and various germs are prevented from infiltrating into the beverage container and from propagating therein. In the ninth aspect, the distance between the circulation passage through which the hot beverage circulates and the connection portion, is such that the hot beverage that is circulating arrives at the connection portion but does not arrive at, or flows little into, the beverage container. In the ninth aspect, further, the hot beverage is circulated to relatively easily sterilize the germs.

According to a tenth aspect of the invention, there is provided a beverage dispenser for supplying a beverage including drinking water from a beverage container through a piping system and a cock, wherein a device for sterilizing by heat the piping system and the cock is provided.

In the tenth aspect, a cooling device for cooling the beverage container is omitted, e.g., a refrigerator is omitted, making it possible to lower the running cost of the dispenser and to lower the cost of production. Further, the dispenser is realized in a small size facilitating the transportation thereof. The piping system and the cock are sterilized by a device that effects the sterilization by heat. When the dispenser is in use, a hygienic beverage can be served. In the tenth aspect, further, not only the piping system but also the cock for supplying the beverage are sterilized by heat, and various germs are prevented from infiltrating into the dispenser through the beverage-pouring port.

According to an eleventh aspect of the invention, there is provided a beverage dispenser for supplying a beverage including drinking water from a beverage container through a piping system and a cock, wherein:

the piping system is provided with a storage tank for storing the beverage;

the storage tank includes at least a cold beverage tank having a cooling device and a hot beverage tank having a heating device; and

a device for sterilizing by heat the piping system, the cold beverage tank, the hot beverage tank and the cock is provided.

In the eleventh aspect, a cooling device for cooling the beverage container is omitted, e.g., a refrigerator is omitted, making it possible to lower the running cost of the dispenser and to lower the cost of production. Further, the dispenser is realized in a small size facilitating the transportation thereof. The piping system, the cock, the cold beverage tank and the hot beverage tank are sterilized by a device that effects the sterilization by heat. When the dispenser is in use, a hygienic hot beverage or cold beverage can be served. In the eleventh aspect, further, not only the piping system but also the cock for supplying the beverage are sterilized by heat, and various germs are prevented from infiltrating into the dispenser through the beverage-pouring port.

A twelfth aspect is as set forth in the eleventh aspect, wherein the device for sterilizing by heat works to circulate the hot beverage in the hot beverage tank.

Namely, in the twelfth aspect, the germs are easily sterilized by circulating the hot beverage.

According to a thirteenth aspect of the invention, there is provided a beverage dispenser for supplying a beverage including drinking water from a beverage container through a piping system and a cock, wherein:

the piping system is provided with a storage tank for storing the beverage;

the storage tank includes at least a cold beverage tank having a cooling device and a hot beverage tank having a heating device;

the cold beverage tank and the hot beverage tank are connected together through a communication pipe; and

a device is provided for sterilizing by heat the piping system, the cock, the cold beverage tank and the hot beverage tank by circulating the hot beverage in the hot beverage tank through the piping system, the cock for pouring the cold beverage, the cold beverage tank and the hot beverage tank using the communication pipe is provided.

In the thirteenth aspect, a cooling device for cooling the beverage container is omitted, e.g., a refrigerator is omitted, making it possible to lower the running cost of the dispenser and to lower the cost of production. Further, the dispenser is realized in a small size facilitating the transportation thereof. The piping system, the cock, the cold beverage tank and the hot beverage tank are sterilized by a device that effects the sterilization by heat. When the dispenser is in use, a hygienic hot beverage or cold beverage can be served. In the thirteenth aspect, further, not only the piping system but also the cock for supplying the beverage are sterilized by heat, and various germs are prevented from infiltrating into the dispenser through the beverage-pouring port.

A fourteenth aspect is as set forth in any one of the eleventh to thirteenth aspects, wherein the cold beverage tank and the hot beverage tank are connected together through a communication pipe via a circulation pump and an electromagnetic valve, and a timer is provided to operate the electromagnetic valve and the circulation pump at regular intervals.

In the fourteenth aspect which uses the timer, the cold beverage in the cold beverage tank and the hot beverage in the hot beverage tank are used in a time zone in which the dispenser is used relatively highly frequently, such as during the day time, and the hot beverage is circulated in a time zone in which the dispenser is used relatively lowly frequently, such as at night, to sterilize the piping system.

A fifteenth aspect is as set forth in any one of the eleventh to fourteenth aspects, wherein absorbing means is provided between the beverage container and the hot beverage tank to absorb a rise in the water level caused by the volumetric expansion of the hot beverage in the hot beverage tank.

In the fifteenth aspect, even when the hot water introduction pipe and/or the check valve of the prior art are not provided for the dispenser, the rise in the water level caused by the volumetric change is absorbed by the absorbing means when the volume of the hot beverage in the hot beverage tank increases by being heated by the heating device. Therefore, the hot beverage is prevented from reversely flowing into the beverage container to an excess degree, and the pressure in the hot beverage tank is maintained within a predetermined range. The absorbing means may be a pipe in the form of, for example, a continuous coil, or may simply be a pipe that is extended between the beverage container and the hot beverage tank. Or, the absorbing means may be another cooling device provided between the beverage container and the hot beverage tank.

A sixteenth aspect is as set forth in any one of the first to fifteenth aspects, wherein the beverage is drinking water.

Namely, in the sixteenth aspect, the drinking water is supplied in a sterilized state.

According to a seventeenth aspect of the invention, there is provided a device for sterilizing a beverage dispenser that supplies a beverage including drinking water from a beverage container through a piping system, wherein the hot beverage is caused to arrive at a connection portion of a beverage container connected to a pipe by utilizing the volumetric expansion of the hot beverage, the beverage container being connected to the hot beverage tank through the pipe.

Namely, in the seventeenth aspect, a cooling device for cooling the beverage container is omitted, e.g., a refrigerator is omitted, making it possible to lower the running cost of the dispenser and to lower the cost of production. Further, the dispenser is realized in a small size facilitating the transportation thereof. The connection portion is sterilized by a device that effects the sterilization by heat. When the dispenser is in use, a hygienic beverage can be served.

According to an eighteenth aspect of the invention, there is provided a device for sterilizing a beverage dispenser that supplies a beverage including drinking water from a beverage container through a piping system and a cock, comprising a heating device for heating the beverage in the hot beverage tank, and a device for circulating the hot beverage in the hot beverage tank heated by the heating device.

Namely, in the eighteenth aspect, a cooling device for cooling the beverage container is omitted, e.g., a refrigerator is omitted, making it possible to lower the running cost of the dispenser and to lower the cost of production. Further, the dispenser is realized in a small size facilitating the transportation thereof. The hot beverage is circulated to easily sterilize the germs. When the dispenser is in use, a hygienic beverage can be served.

According to the first aspect of the invention, a common effect is exhibited in that when the dispenser is in use, a hygienic beverage can be served.

According to the second aspect, the piping system, cold beverage tank and hot beverage tank are sterilized by the device that effects the sterilization by heat.

According to the third aspect, the connection portion is sterilized by the hot beverage preventing various germs from infiltrating into the beverage container and from propagating therein.

According to the fourth aspect, the hot beverage is circulated to relatively easily sterilize the connection portion.

According to the fifth aspect, the piping system, cold beverage tank, hot beverage tank and connection portion are sterilized by the device that effects the sterilization by heat.

According to the sixth aspect, the hot beverage is prevented from reversely flowing into the beverage container to an excess degree, and the pressure in the hot beverage tank is maintained to lie in a predetermined range.

According to the seventh aspect, the cold beverage in the cold beverage tank and the hot beverage in the hot beverage tank are used in a time zone in which the dispenser is used relatively highly frequently, and the hot beverage is circulated in a time zone in which the dispenser is used relatively lowly frequently to sterilize the piping system.

According to the eighth aspect, the piping system, cock, cold beverage tank, hot beverage tank and connection portion are sterilized by the device that effects the sterilization by heat.

According to the ninth aspect, the connection portion is sterilized by the hot beverage preventing various germs from infiltrating into the beverage container and from propagating therein.

According to the tenth aspect, the piping system and the cock are sterilized by the device that effects the sterilization by heat.

According to the eleventh aspect, the piping system, the cock, the cold beverage tank and the hot beverage tank are sterilized by the device that effects the sterilization by heat.

According to the twelfth aspect, the hot beverage is circulated to easily sterilize the germs.

According to the thirteenth aspect, the piping system, the cock, the cold beverage tank and the hot beverage tank are sterilized by the device that effects the sterilization by heat.

According to the fourteenth aspect, the cold beverage in the cold beverage tank and the hot beverage in the hot beverage tank are used in a time zone in which the dispenser is used relatively highly frequently, and the hot beverage is circulated in a time zone in which the dispenser is used relatively lowly frequently to sterilize the piping system.

According to the fifteenth aspect, the hot beverage is prevented from reversely flowing into the beverage container to an excess degree, and the pressure in the hot beverage tank is maintained to lie in a predetermined range.

According to the sixteenth aspect, the drinking water is supplied in a sterilized state.

According to the seventeenth aspect, the connection portion is sterilized by the device that effects the sterilization by heat.

According to the eighteenth embodiment, the hot beverage is circulated to easily sterilize the germs.

### BRIEF DESCRIPTION OF THE DRAWINGS

FIG. 1 is a diagram schematically illustrating a beverage dispenser according to the present invention;

FIG. 2A is a sectional view illustrating, on an enlarged scale, a cold water-pouring cock when it is being used;

FIG. 2B is a sectional view illustrating, on an enlarged scale, the cold water-pouring cock during the circulation;

FIG. 3A is a sectional view illustrating, on an enlarged scale, a hot water-pouring cock and a cold water-pouring cock;

FIG. 3B is a other sectional view illustrating, on an enlarged scale, another hot water-pouring cock and another cold water-pouring cock;

FIG. 4 is a sectional view illustrating, on an enlarged scale, a portion of the dispenser according to the present invention;

FIG. 5A is a partial perspective view illustrating the dispenser according to the present invention;

FIG. 5B is a partial perspective view illustrating a state where a drinking water container is being installed;

FIG. 5C is a partial perspective view illustrating a state where the drinking water container is being installed; and

FIG. 6 is a view schematically illustrating a conventional drinking water dispenser.

### BEST MODE FOR CARRYING OUT THE INVENTION

Embodiments of the invention will now be described with reference to the accompanying drawings. In the drawings, the same members are denoted by the same reference numerals. For easy understanding, the scales of these drawings are suitably varied.

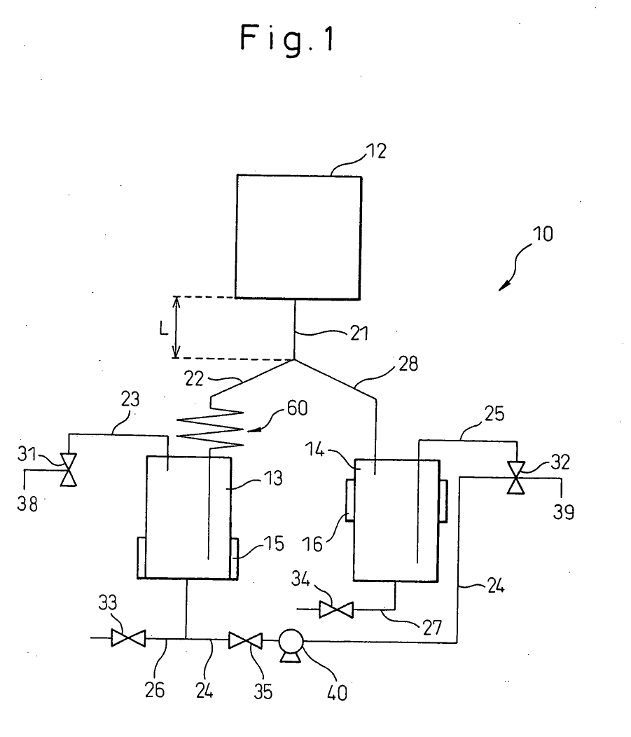

FIG. 1 is a view schematically illustrating a beverage dispenser according to the present invention. A drinking water container 12 filled with drinking water as a beverage is a bag-in-box type container which is mounted on a dispenser 10 by cutting an end of a pouring tube that is formed being connected to an inner bag (not shown) of the bag-in-box type container. The container filled with the drinking water may be any kind of container capable of absorbing the volumetric expansion of the drinking water due to the heating and is intimately sealed. If the inner bag of the bag-in-box type container is of the type provided with a spout, then, the container may be connected to the dispenser through the spout and a connection fitting.

Referring to FIG. 1, a common pipe 21 extending from the drinking water container 12 is branched into a branched pipe 22 and a branched pipe 28, which are connected to two kinds of storage tanks, i.e., to a hot water tank 13 and to a cold water tank 14. The drinking water supplied from the drinking water container 12 into the hot water tank 13 is heated by a heating device 15 to a suitable temperature which may be, for example, about 70° C. or higher. Similarly, the drinking water supplied from the drinking water container 12 into the cold water tank 14 is cooled by a cooling device 16 down to about 4° C. to about 10° C. By operating a hot water-pouring cock 31 provided in the pipe 23 connected to the hot water tank 13, hot water is poured from the pouring port 38. Further, by operating a cold water-pouring cock 32 provided in the pipe 25 connected to the cold water tank 14, cold water is poured from the pouring port 39.

As will be understood from FIG. 1, the branched pipe 22 for pouring the drinking water into the hot water tank 13 extends deep into the hot water tank 13, e.g., extends to not less than 75% of the depth thereof, whereas the pipe 23 for pouring the drinking water out of the hot water tank 13 is extending shallowly into the hot water tank 13, e.g., up to not more than 25% of the depth. The hot water circulates by convection toward the upper part of the hot water tank 13 (upper side in FIG. 1). Therefore, the hot water is efficiently poured from the hot water tank 13.

The branched pipe 28 for pouring the drinking water into the cold water tank 14 extends only shallowly into the cold water tank 14, e.g., into a depth of not more than 25%, whereas the pipe 25 for pouring the drinking water out of the cold water tank 14 is extending deep into the cold water tank 14, e.g., up to not less than 75% of the depth. The cold water circulates by convection toward the bottom of the cold water tank 14 (lower side in FIG. 1). Therefore, the cold water is efficiently poured from the cold water tank 14.

An air vent (not shown) is formed in the wall near the upper part of the cold water tank 14, and bubbles that have arrived at the upper part of the cold water tank 14 enter into the pipe 25 through the air vent, and are drained through the cold water cock 32.

As will be understood from FIG. 1, a communication pipe 24 extending from the hot water tank 13 is connected to the cold water-pouring cock 32 of the pipe 25, the communication pipe 24 being provided with a circulation valve 35 and a circulation pump 40. The circulation valve 35 is an electromagnetic valve that is operated by, for example, a solenoid. As shown, further, a pipe 26 provided with a drain valve 33 is extending from a communication pipe 24 and, similarly, a pipe 27 provided with a drain valve 34 is extending from the cold water tank 14.

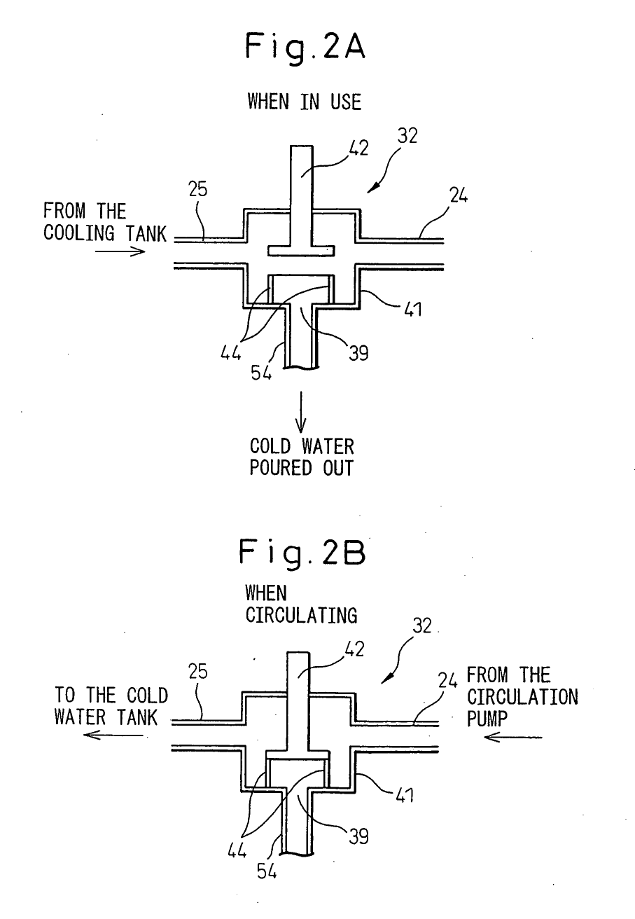

FIG. 2A is a sectional view illustrating, on an enlarged scale, the cold water-pouring cock when it is being used, and FIG. 2B is a sectional view illustrating, on an enlarged scale, the cold water-pouring cock during the circulation. As shown in these drawings, the cold water-pouring cock 32 includes a valve seat 44 provided in a casing 41 and a valve body 42 that comes into contact therewith. The valve body 42 is usually in contact with the valve seat 44 causing the cold water-pouring cock 32 to be closed. When a user pushes down a lever (not shown), however, the valve body 42 separates away from the valve seat 44, and the cold water-pouring cock 32 is opened. As will be described later, further, the hot water-pouring cock 31, too, is constituted nearly in the same manner as the cold water-pouring cock 32. The valve body 42 can be opened and closed even by a separately provided actuator (not shown) in addition to being operated by the user through the lever. Concretely speaking, if the valve body 42 and valve body 47 remain opened during the heat-sterilization by circulating the hot water, the user of the dispenser may suffer a burn. During the sterilization by heat, therefore, the valve body 42 and valve body 47 is closed. Even after the sterilization by heating, the valve body 42 and valve body 47 is controlled to remain closed until the temperature of the cold water tank 14 decreases down to 30° C. or lower so that the user of the dispenser will not suffer a burn.

Referring to FIG. 2A, when the dispenser 10 is in use, the user pushes the lever down to open the valve body 42. Therefore, the cold water in the cold water tank 14 flows into the cold water-pouring cock 32 through the pipe 25, and is poured from the pouring port 39 of the cold water-pouring cock 32. When in use, the circulation valve 35 (see FIG. 1) in the communication pipe 24 is closed. Therefore, the cold water that may flow into part of the communication pipe 24 shown in FIG. 2A during the use does not flow up to the side of the hot water tank 13 passing through the circulation valve 35.

When the dispenser 10 is not in use, i.e., when the hot water-pouring cock 31 and the cold water-pouring cock 32 are closed, on the other hand, the circulation valve 35 in the communication pipe 24 shown in FIG. 1 is opened and the circulation pump 40 is driven. Therefore, the hot water in the hot water tank 13 passes through the communication pipe 24 and arrives at the cold water-pouring cock 32 as shown in FIG. 2B. The body 42 at this moment is in contact with the valve seat 44 and the cold water-pouring cock 32 is closed. Therefore, the hot water that flowed into the cold water-pouring cock 32 from the communication pipe 24 is not poured from the pouring port 39 but flows into the cold water tank 14 through the pipe 25. The hot water that flowed into the cold water tank 14 so circulates as to return into the hot water tank 13 through the branched pipe 28 and the branched pipe 22. As described above, the hot water in the hot water tank 13 has been heated at a temperature of not lower than 55° C. and is capable of accomplishing the heat-sterilization. Therefore, the hot water that is circulating works to heat-sterilize the communication pipe 24, cold water-pouring cock 32, pipe 25, cold water tank 14, branched pipe 28, branched pipe 22 and hot water tank 13. When the hot water tank 13 is heated at a temperature in excess of 95° C., it becomes probable that the hot water reversely flows to the drinking water container 12. Therefore, the operation of the heating device 15 is stopped or controlled by a separately provided control device (not shown).

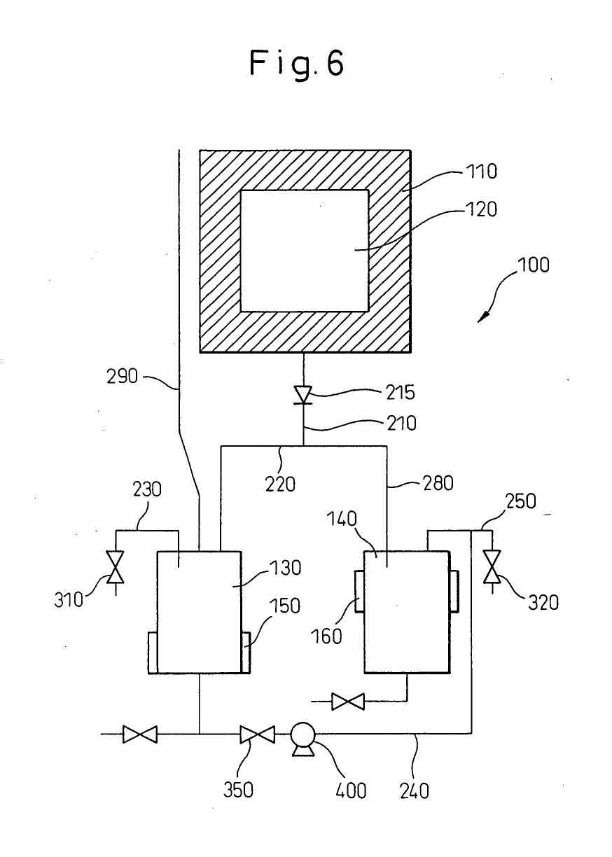

The present invention uses no cooling device for cooling the drinking water container 12, i.e., uses no refrigerator (see refrigerator 110 shown in FIG. 6). Therefore, the drinking water in the drinking water container 12 is maintained at normal temperature, and it is probable that various germs that have infiltrated through the pouring ports 38 and 39 into the drinking water container 12 may propagate in the drinking water container 12. However, as the hot water is circulated to heat-sterilize the communication pipe 24, cold water-pouring cock 32, pipe 25, cold water tank 14, branched pipe 28, branched pipe 22 and hot water tank 13, it is made possible to prevent various germs from infiltrating into the drinking water container 12. When the dispenser 10 is in use, therefore, hygienic hot water or cold water can be served. During the heat-sterilization, it is preferable to discontinue the operation of the cooling device 14 for the cold water tank 14. This avoids the temperature of the hot water being circulated from becoming lower than the sterilization temperature. When the hot water is to be circulated, further, the valve body 42 shown in FIG. 2B may be suitably elevated by an actuator (not shown), so that the hot water flows to the pouring port 39 of the cold water-pouring cock 32 to heat-sterilize the pouring port 39.

Further, the circulation valve 35 and the circulation pump 40 are connected to a timer (not shown). Upon setting the timer, it is made possible to open the circulation valve 35 and to drive the circulation pump 40 after the passage of a predetermined period of time and, then, to close the circulation valve 35 and to halt the circulation pump 40 after the passage of another predetermined period of time. The dispenser 10 of this invention is, in many cases, installed in offices and in general households, and is frequently used in a predetermined time zone such as during the day time but the frequency of use sharply drops in another predetermined time zone such as at night. Therefore, the timer may be so set that the hot water circulates in only a time zone where the frequency of use is low, such as at night to heat-sterilize the dispenser 10 in the time zone where the frequency of use is low without spoiling the convenience for the users.

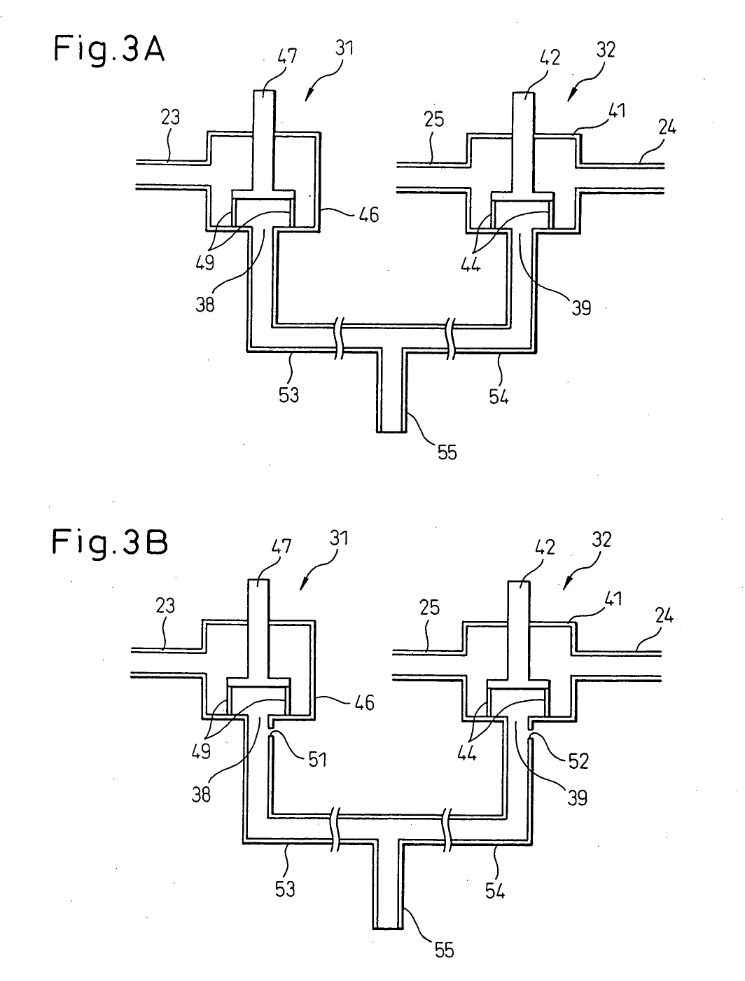

FIGS. 3A and 3B are sectional views illustrating, on an enlarged scale, a hot water-pouring cock and a cold water-pouring cock. Referring to FIG. 3A, the hot water-pouring cock 31 is constituted in the same manner as the cold water-pouring cock 32, and includes a valve body 47 in the casing 46 and a valve seat 49 that comes in contact therewith. As shown, the pouring port 38 of the hot water-pouring cock 31 is communicated with a pipe 53, and the pouring port 39 of the cold water-pouring cock 32 is communicated with a pipe 54. The pipes 53 and 54 are further communicated with a common pouring port 55. When the dispenser 10 is in use, therefore, both the hot water from the hot water-pouring cock 31 and the cold water from the cold water-pouring cock 32 are poured from the common pouring port 55.

When in use, the temperature is relatively high near the hot water-pouring cock 31. Therefore, various germs infiltrated through the common pouring port 55 are heat-sterilized near the hot water-pouring cock 31. The temperature, on the other hand, is relatively low near the cold water-pouring cock 32. Therefore, various germs adhered near the cold water-pouring cock 32 are not heat-sterilized. In this invention, however, the pipe 53 for supplying the hot water and the pipe 54 for supplying the cold water are communicated with each other. Therefore, the hot water poured from the common pouring port 55 through the pipe 53 flows into the pipe 54. When the hot water is poured, therefore, the pipe 54 for supplying the cold water is heat-sterilized simultaneously. When in use, therefore, various germs are prevented from entering into the cold water-pouring cock 32.

Referring to FIG. 3B, further, a hole 51 may be formed in the pipe 53 relatively close to the pouring port 38 of the hot water-pouring cock 31, and a hole 52 may be formed in the pipe 54 relatively close to the pouring port 39 of the cold water-pouring cock 32. These holes 51 and 52 are much smaller than the pipes 53 and 54. When the dispenser 10 is in use, therefore, the hot water and the cold water passing through the pipe 53 and the pipe 54, do not flow out through the holes 51 and 52. When the hot water and the cold water are poured from the common pouring port 55, the air goes out of the pipes 53 and 54 through the holes 51 and 52. Therefore, the hot water and the cold water in the pipes 53 and 54 easily arrive at the pipes 53 and 54. Thereafter, the air enters into the pipes 53 and 54 through the holes 51 and 52, whereby the hot water and the cold water in the pipes 53 and 54 are pushed by the air and flow out from the common pouring port 55. The hot water or the cold water is prevented from staying in the pipes 53, 54 or from staying in the common pouring port 55. Therefore, the dispenser of this invention prevents various germs from infiltrating into the hot water or the cold water staying in the pipes 53, 54 and in the common pouring port 55.

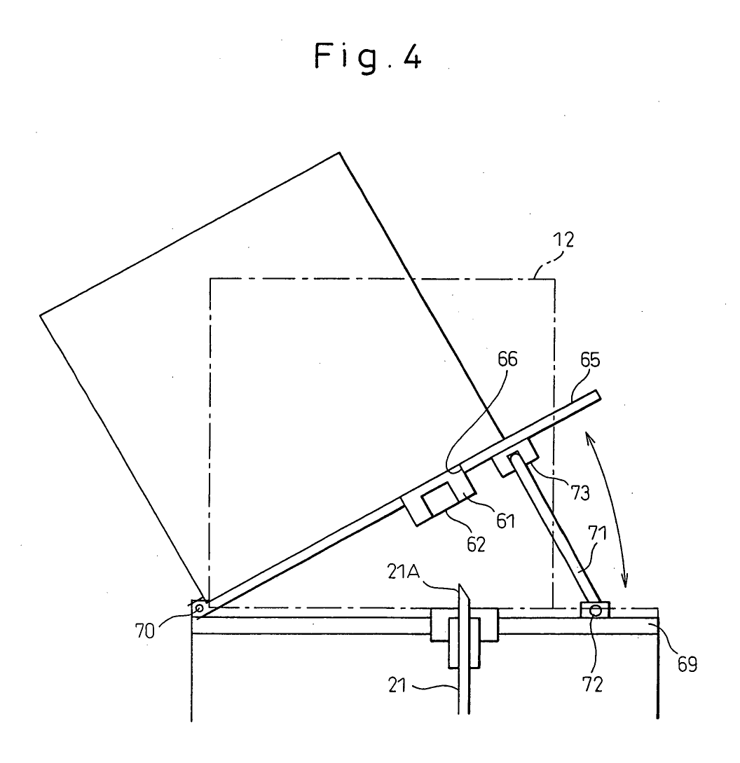

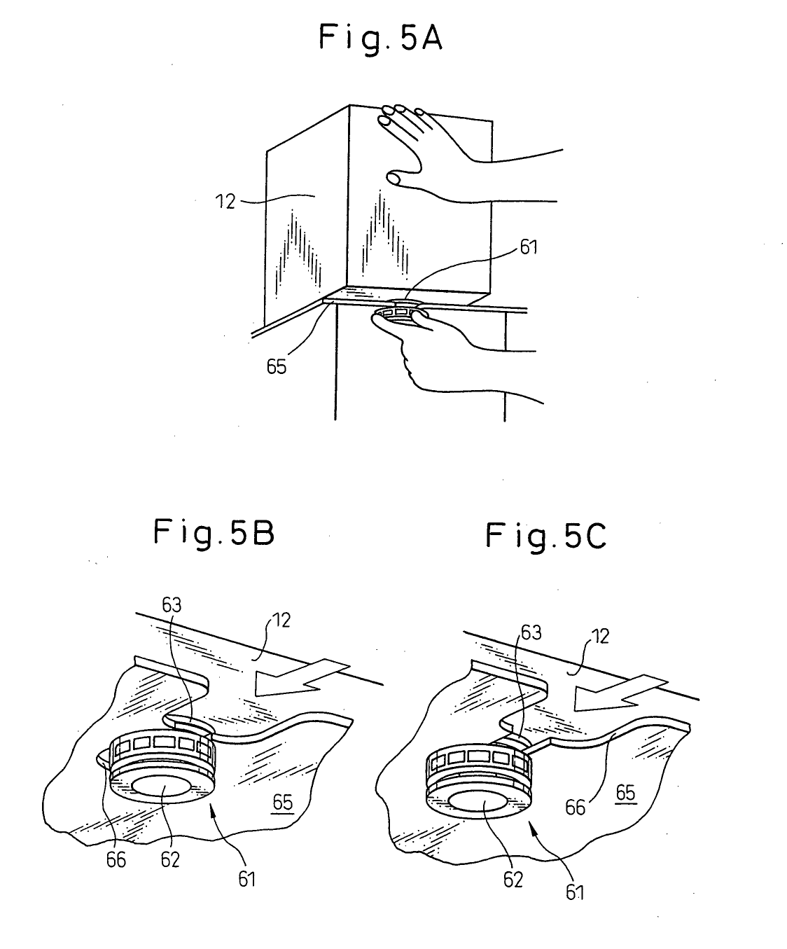

Next, described below is how to install the drinking water container 12 in the body of the dispenser 10. FIG. 4 is a view illustrating, on an enlarged scale, a portion of the dispenser according to the invention, FIG. 5A is a is a partial perspective view illustrating the dispenser according to the invention, and FIGS. 5B and 5C are partial perspective views illustrating a state where the drinking water container is being installed. As described above, the drinking water container 12 is a sealed container of the bag-in-box type. A pouring port 62 formed being coupled to the inner bag is connected to a common pipe 21 on the side of the dispenser. Referring to FIG. 4, the pouring port 62 is formed in the neck portion 61, and a seal that can be peeled off is stuck to the top panel of the neck portion. A seal film is provided at the inner part in the neck portion to maintain the drinking water filled therein aseptic. When connected to the common pipe 21 on the side of the dispenser as shown in FIG. 5A, the drinking water container 12 is installed on a rack plate 65 of the dispenser in a state where the seal is peeled off the top panel in the neck portion of the pouring port.

Referring to FIG. 5B, a recessed portion 66 is formed in the rack plate 65. To install the drinking water container 12, first, the neck portion 61 of the drinking water container 12 is partly inserted in the recessed portion 66. At this moment, a groove portion 63 formed in the neck portion 61 is fitted to the rack plate 65. Next, referring to FIG. 4, again, the rack plate 65 is slightly pivoted about a hinge 70 so as to tilt the rack plate 65 on which the drinking water container 12 is arranged, and a stand 71 that pivots about a hinge 72 is brought into engagement with a stand-engaging portion 73 provided on the rack plate 65 thereby to maintain the rack plate 65 aslant. Referring next to FIG. 5C, the neck portion 61 of the drinking water container 12 is completely pushed into the rear. Then, the neck portion 61 completely pushed into the recessed portion 66 is arranged at a position corresponding to a sharp end 21A of the common pipe 21 attached to the rack plate 69 of the dispenser 10. Next, the rack plate 65 is pivoted to a horizontal position as represented by a dot-dash chain line in FIG. 4. At this moment, the rack plate 65 is pivoted in a direction to be further lifted up aslant, so that the stand 71 is disengaged from the stand-engaging portion 73. The stand 71 returns by itself to the state of before being pivoted. Therefore, the sharp end 21A of the common pipe 21 enters into the pouring port 62 of the neck portion 61 and pierces the seal film in the neck portion 61. Due to its own weight, therefore, the drinking water in the drinking water container 12 flows into the common pipe 21. The drinking water container 120 of the dispenser 100 according to the prior art is arranged in the refrigerator 110 (see FIG. 6), and the rack plate provided with the drinking water container 120 cannot be pivoted. The dispenser 10 of the present invention, on the other hand, is not provided with the refrigerator 110. Upon simply pivoting the rack plate 65 provided with the drinking water container 12, therefore, the seal film in the neck portion 61 can be easily pierced by utilizing the weight of the drinking water container and of the drinking water in the drinking water container, making it very easy to install the drinking water container 12.

As will be understood with reference again to FIG. 1, when the drinking water in the hot water tank 13 is heated by the heating device 15, its volume expands. In the prior art, the hot water introduction pipe 290 is provided to absorb the rise in the water level caused by the volumetric expansion (see FIG. 6). According to the present invention, the dispenser 10 is not provided with the hot water introduction pipe, and the common pipe 21 of the dispenser 10 is not provided with the check valve. Therefore, the hot water that circulates may partly flow into the common pipe 21. The pipe 22, pipe 28 and common pipe 21 are connected in an inverse Y-shape, and the drinking water stays in the connected portion in order to prevent the propagation of various germs. Further, the dispenser 10 of this invention is not provided with a refrigerator for cooling the drinking water container 12. Therefore, the length L of the common pipe 21 must be such that the expanded volume of the hot water does not flow, or flows only a little, into the drinking water container 12 through the common pipe 21, so that various germs will not propagate in the drinking water container 12. At the time of installing the drinking water container 12, on the other hand, various germs may infiltrate when the sharp end 21A of the common pipe 21 pierces the seal film in the neck portion 61. It is therefore preferably that the neck portion 61 is heat-sterilized when the hot water circulates. Namely, it is preferably that the length L of the common pipe 21 is such that part of the hot water flowing through the pipe 28 and pipe 22 due to the expansion of the hot water arrives at the neck portion 61 only of the drinking water container 12. This makes it possible to heat-sterilize the neck portion 61 of the drinking water container 12.

When in use, further, the temperature of the drinking water in the hot water tank is elevated by being heated by the heating device 15, and the volume expands. In the present invention, a portion 60 for absorbing the rise of water level due to the volumetric expansion is arranged between the drinking water container 12 and the hot water tank 13 in FIG. 1. The portion 60 for absorbing the rise of water level due to the volumetric expansion in FIG. 1 is formed in the shape of a continuous coiled pipe. When the dispenser 10 is in use, the circulation valve 35 is closed. Therefore, part of the drinking water of which the volume is expanded by being heated by the heating device 15, i.e., the hot water of an amount that is expanded, can flow into the portion 60 for absorbing the rise of water level. The drinking water in the portion 60 for absorbing the rise of water level reversely flows into the drinking water container 12 due to the volumetric expansion. As a result of the volumetric expansion, the hot water is spontaneously cooled in the portion 60 for absorbing the rise of water level, and the volume of an amount that has expanded contracts. In the present invention, therefore, the hot water in the hot water tank 13 is prevented from reversely flowing into the drinking water container 12.

The portion 60 for absorbing the rise of water level caused by a change in the volume of the hot water shown in FIG. 1 is a pipe in the form of a continuous coil. However, a pipe between the drinking water container and the hot water tank may simply be extended so as to work as the portion 60 for absorbing the rise of water level. If the pipe 22 is introduced into the hot water tank 13 from the side surface close to the bottom of the hot water tank 13, then, the pipe system need not be unnecessarily expanded. Besides, another cooling device (not shown) provided between the drinking water container 12 and the hot water tank 13 may be used as the portion 60 for absorbing the rise of water level, so that the hot water is cooled and its volume is reliably reduced. Moreover, some of the above-mentioned embodiments may be suitably combined together without departing from the scope of the invention.

### EXAMPLE

The drinking water container 12 was installed in the dispenser 10 of the invention in a laboratory, the drinking water was charged into the piping system according to a predetermined procedure, and the heating device 15 and the cooling device 16 were driven. When the operation of the dispenser 10 was stabilized after the passage of a predetermined period of time or, here, when the temperature of the drinking water in the dispenser 10 has reached about 70 to 80° C. after the passage of 30 minutes, the hot water-pouring cock 31 of the dispenser 10 was operated to pour the hot water into three sterilizing Erlenmeyer flasks of a capacity of 500 ml. Similarly, the cold water-pouring cock 32 was operated to pour the cold water into another three sterilizing Erlenmeyer flasks of a capacity of 500 ml. The hot water and cold water samples poured into the Erlenmeyer flasks were quickly tested for microorganisms.

After the above sampling, the dispenser 10 is maintained in a state where it is usually used, and the cold water and the hot water in amounts that would be usually used were poured out every day through the hot water-pouring cock 31 and the cold water-pouring cock 32.

The microorganism testing was conducted after the operation of the dispenser 10 was stabilized (0th day), on the 4th day, 7th day, 14th day and 21st day after the operation of the dispenser 10 was stabilized. The cold water samples and the hot water samples were examined for their numbers of living germs and were tested for their coliform group as described below.

To examine the number of living germs, 3 g/l of a Trypticase Soy Broth (manufactured by Nihon Pecton-Deckinson Co.) and 15 g/l of agar were dissolved in distilled water, and were sterilized with high-pressure steam at a temperature of 121° C. for 15 minutes. After sterilization, the dissolved matter was poured in an amount of about 15 ml into the sterilized plastic laboratory dishes (diameter of 90 mm, depth of 15 mm) to prepare 1/10 TSA culture media which were agar flat board culture media.

Next, the cold water samples in an amount of 1 ml (hereinafter referred to as “sample A”), in an amount of 10 ml (hereinafter referred to as “sample B”), and in an amount of 100 ml (hereinafter referred to as “sample C”) were passed through membrane filters. The membrane filters were placed on the 1/10 TSA culture media, and were cultured at a temperature of 28° C. for 5 days to examine the numbers of living germs (membrane filtering method). Similarly, the hot water samples in an amount of 100 ml (hereinafter referred to as “sample D”), in an amount of 100 ml (hereinafter referred to as “sample E”), and in an amount of 100 ml (hereinafter referred to as “sample F”) were passed through membrane filters. The membrane filters were placed on the 1/10 TSA culture media, and were cultured at a temperature of 28° C. for 5 days to examine the numbers of living germs (membrane filtering method). Table 1 shows the examined results of the numbers of living germs. In Table 1, the number of living germs is per 100 ml (cells/100 ml).

TABLE 1
The Number of Living Germs (cells/100 ml)

| The Number | | | | | | |
|---|---|---|---|---|---|---|
| of Days from | Samples from the Cold | | | Samples from the Hot | | |
| the Start of | Water-pouring Cock | | | Water-pouring Cock | | |
| the Test | Sample | Sample | Sample | Sample | Sample | Sample |
| (Day) | A | B | C | D | E | F |
| 0 | 0 | 0 | 0 | 0 | 0 | 0 |
| 4 | 0 | 0 | 0 | 0 | 0 | 0 |
| 7 | 0 | 0 | 0 | 0 | 0 | 0 |
| 14 | 0 | 0 | 0 | 0 | 0 | 0 |
| 21 | 0 | 0 | 0 | 0 | 0 | 0 |

To test the coliform group, further, the cold water sample and the hot water sample each in an amount of 50 ml were poured into a coliform group testing culture medium (Pro-media XM-50, manufactured by Ermex Co.). Next, cold water samples were poured from the cold water cock into three culture media (sample G, sample H, sample I). Similarly, hot water samples were poured from the hot water cock into three culture media (sample J, sample K, sample L). These samples were cultured at a temperature of 35° C. for 18 hours to 24 hours to judge the results. Table 2 shows the results of the test of

TABLE 2
Coliform Group Test

| The Number of | | | | | | |
|---|---|---|---|---|---|---|
| Days from | | | | | | |
| the Start of | Samples from the Cold | | | Samples from the Hot | | |
| the Test | Water-pouring Cock | | | Water-pouring Cock | | |
| (Day) | Sample G | Sample H | Sample I | Sample J | Sample K | Sample L |
| 0 | Negative | Negative | Negative | Negative | Negative | Negative |
| 4 | Negative | Negative | Negative | Negative | Negative | Negative |
| 7 | Negative | Negative | Negative | Negative | Negative | Negative |
| 14 | Negative | Negative | Negative | Negative | Negative | Negative |
| 21 | Negative | Negative | Negative | Negative | Negative | Negative |

As shown in Table 1, the number of living germs was zero in both the cold water samples (collected from the cold water-pouring cock) and the hot water samples (collected from the hot water-pouring cock) after the operation of the dispenser 10 was stabilized until 21 days have passed. As shown in Table 2, further, the results of the test of coliform group were negative in both the cold water samples and the hot water samples after the operation of the dispenser 10 was stabilized until 21 days have passed.

From the examination of the number of living germs and the testing of coliform group, therefore, it was learned that hygienic drinking water can be supplied even when a refrigerator is not used for the drinking water container. The above dispenser makes it possible to lower the running cost and the production cost, and can be realized in a small size, facilitating the transportation thereof. Besides, the dispenser can be installed in a place where it was difficult to install the dispenser for supplying hygienic drinking water, such as in general households.

Although the invention has been shown and described with exemplary embodiments thereof, it should be understood by those skilled in the art that the foregoing and various other changes, omissions and additions may be made therein and thereto without departing from the spirit and the scope of the invention.

1.-10. (canceled)

11. A beverage dispenser for supplying a beverage including drinking water from a beverage container through a piping system and a cock, wherein:
said piping system is provided with a storage tank for storing the beverage;
said storage tank includes at least a cold beverage tank having a cooling device and a hot beverage tank having a heating device; and
a device for sterilizing by heat said piping system, said cold beverage tank, said hot beverage tank and said cock is provided.

12. A beverage dispenser according to claim 11, wherein said device for sterilizing by heat works to circulate the hot beverage in the hot beverage tank.

13. (canceled)

14. A beverage dispenser according to claim 11, wherein said cold beverage tank and said hot beverage tank are connected together through a communication pipe via a circulation pump and an electromagnetic valve, and a timer is provided to operate said electromagnetic valve and said circulation pump at regular intervals.

15. A beverage dispenser according to claim 11, wherein absorbing means is provided between said beverage container and said hot beverage tank to absorb a rise in the water level caused by the volumetric expansion of the hot beverage in said hot beverage tank.

16-18. (canceled)
